(19) United States
(12) Patent Application Publication
(10) Pub. No.: US 2007/0156246 A1
(43) Pub. Date: Jul. 5, 2007

(54) Shoulder joint prosthetic system

(76) Inventors: Jayantilal Mohanlal Meswania, Colney Heath (GB); Simon Lambert, Kings Langley (GB); Ian Bayley, Harrow (GB)

Correspondence Address: Bradley N Ruben, Suite 5A, 463 First Street, Hoboken, NJ, 07030-1859, US

(21) Appl. No.: 10/569,888

(22) PCT Filed: Aug. 31, 2004
(86) PCT No.: PCT/GB04/03721

§ 371 Date: Oct. 23, 2006

(30) Foreign Application Priority Data
Aug. 29, 2003 (GB) 0320287.6

(51) Int. Cl. A61F 2/40

(52) U.S. Cl. 623/19.12; 623/19.13

(57) ABSTRACT

In a first aspect the present invention provides a glenoid prosthesis assembly for use in shoulder joint arthroplasty to address conditions where deterioration of the rotator cuff mechanism of the shoulder joint is severe, the assembly comprising a first shell component to be affixed to a scapula by fixing means, and a smaller second component adapted to nest within the first component and having an articulation surface for articulating with a humeral head, the first component being over-sized relative to the glenoid surface of a scapula whereby the first component may be first affixed to at least two of the glenoid, acromion and coracoid processes of the scapula in use by the fixing means and the second component may be cemented in place within the first component allowing initial adjustability in the poise of the second component relative to the first component. Amongst other aspects the invention also provides a modular proximal humeral prosthesis system for use in shoulder joint arthroplasty which enables the proximal humeral prosthesis to be changed between anatomical and reverse of anatomical configurations to address differing degrees of shoulder joint deterioration.

### FIELD OF THE INVENTION

The present invention concerns improvements relating to a shoulder joint prosthetic system and to components suitable for use in the system.

### BACKGROUND TO THE INVENTION

Of the joints of the human body that may be repaired with prosthetic components, the shoulder joint is particularly awkward to satisfactorily address with mass produced prosthetic components. In part this is because the joint is defined on the scapula side by three separate processes of the scapula and of which the critical abutment surface for the corresponding ball of the humerus is a thin-walled process of the scapula defining the glenoid cavity. The soft tissue linkages comprising muscles, ligaments and tendons forming the so-called rotator cuff mechanism between the scapula and the humerus play a critical part in the support and extent of manoeuvrability of the shoulder and have a number of disparate attachment points. Depending upon the nature of the disease or other cause of damage to the shoulder, the condition of the proximal humerus may be good or poor and a number of prosthesis components exist for replacing/repairing the proximal humerus such as illustrated in, for example, U.S. Pat. No. 5,741,335 and U.S. Pat. No. 5,549,682. Such devices generally have a stem that is tapered and which is lodged into the resected proximal humerus and which carries a part-spherical articulation surface to co-operate with the glenoid cavity of the scapula. The part-spherical surface of the prosthetic component may be integrated with the shaft or may be a separate modular component that is assembled on to the shaft prior to use.

For many damaged shoulder joint conditions, however, the damage to the humerus is the lesser problem to be dealt with. The extent of damage to the processes of the scapula and especially to the glenoid cavity may vary greatly and when extreme can make repair of the joint extremely difficult. Futhermore, the condition of bearing surfaces of the bone such as the glenoid may well deteriorate over time following installation of a prosthesis, through wear and tear or through ongoing effects of chronic disease, including cancer or osteoporosis.

Prior attempts to address damage to the glenoid include prosthetic components as disclosed in U.S. Pat. No. 4,550,450, U.S. Pat. No. 4,919,669 and U.S. Pat. No. 4,693,723.

However, none of these known shoulder joint prostheses/prosthetic systems is fully versatile, none enables repair of very severely damaged glenoid bearing surfaces, none provide for optimal soft tissue (muscle/ligament) attachment to the proximal humeral prosthetic component and none of the systems make proper allowance for progressive deterioration of the bone of the joint so that progressive modifications may be made as the joint deteriorates.

### SUMMARY OF THE INVENTION

According to a first aspect of the present invention there is provided a glenoid prosthesis assembly for use in shoulder joint arthroplasty comprising a first, suitably shell/cup-shaped, component to be affixed to a scapula by fixing means, the fixing means suitably comprising bolts, screws or the like, and a smaller second, suitably shell/cup-shaped, component adapted to nest within the first component and having an articulation surface for articulating with a humeral head, the first component being over-sized relative to the glenoid surface of a scapula whereby the first component may be first affixed to at least two of the glenoid, acromion and coracoid processes of the scapula in use by the fixing means and the second component may be cemented in place within the first component allowing initial adjustability in the poise of the second component relative to the first component.

Suitably at least one of the first and second components has on its surface that mounts to the other component grooves to facilitate cement distribution for cementing the second component in place.

Particularly preferably the first cup-shaped component is affixed in use to each one of the glenoid, acromion and coracoid processes of the scapula and preferably for this purpose has at least one respective screw/bolt hole therethrough located to correspond to the relative location of each of the glenoid, acromion and coracoid processes of the scapula.

According to a second aspect of the present invention there is provided a proximal humeral prosthesis for use in shoulder joint arthroplasty which comprises a humeral head for articulation with a glenoid articular surface and a stem to fit into a resected humerus, the prosthesis being formed to replace substantially the whole of the upper end of a proximal humerus including the neck and one or both tubercles and having a projecting flange or fin arrangement thereon to which soft tissues of the shoulder may attach and anchor.

The flange or fin arrangement is advantageously configured to provide a first length that extends transversely of the axis of the stem and a second length that extends at an angle to the first which is more aligned with the stem to provide anchoring against forces in more than one axis of articulation of the humerus at the shoulder. The first length of fin or flange is suitably configured to anchor against forces for lifting of the humerus/arm and the second length of fin or flange is suitably configured to anchor against forces for rotation of the humerus/arm about its axis. Preferably the first length of fin or flange is separated from the second length by a gap for passage of nerves or other tissues.

According to a further aspect of the present invention there is provided a modular proximal humeral prosthesis system for use in shoulder joint arthroplasty which comprises an upper proximal humeral prosthesis part for articulation with a glenoid mounted prosthesis and a proximal humeral prosthesis stem to fit into a resected humerus, the upper part being demountably mounted to the stem in use, there being a range of alternative upper parts whereof: a first upper proximal humeral prosthesis part has thereon or coupled thereto in use a head component that articulates with a glenoid mounted prosthesis and defining a convex humeral head articulating surface to co-operate with a glenoid mounted component whereof the articulating surface is concave (anatomical configuration); and a second upper proximal humeral prosthesis part which has thereon or coupled thereto in use a head component that articulates with a glenoid mounted prosthesis and defining a concave articulating surface to co-operate with a glenoid mounted component whereof the articulating surface has a convex rounded form (reverse of anatomical configuration), whereby the proximal humeral prosthesis may be changed between anatomical and reverse of anatomical configurations.

According to a yet further aspect of the present invention there is provided a modular proximal humeral prosthesis for use in shoulder joint arthroplasty to articulate with a glenoid mounted prosthesis and which comprises an upper part, a head component that assembles to the upper part and articulates with the glenoid mounted prosthesis, and a stem to fit into a resected humerus, the upper part of the prosthesis being formed to replace substantially the whole of the upper end of a proximal humerus including the neck and one or both tubercles, having a substantially hemispherical or rounded outer form with a formation, suitably a recess or projection, at an inner face thereof that faces in use toward the glenoid mounted prosthesis, the formation fitting in use to a coupling part of the head component. One head component that articulates with the glenoid mounted prosthesis suitably defines a convex humeral head articulating surface while another defines a concave articulating surface.

According to a further aspect of the invention there is provided a modular proximal humeral prosthesis system for use in shoulder joint arthroplasty to articulate with a glenoid mounted prosthesis and which comprises an upper part and a stem to fit into a resected humerus, and a head component that assembles to the upper part and articulates with the glenoid mounted prosthesis, there being a range of alternative head components whereof: a first head component has a convex humeral head articulating surface to co-operate with a glenoid mounted component whereof the articulating surface is concave (anatomical configuration); and a second head component has a concave articulating surface to co-operate with a glenoid mounted component whereof the articulating surface has a convex rounded form (reverse of anatomical configuration), whereby the proximal humeral prosthesis may be changed between anatomical and reverse of anatomical configurations. Suitably the proximal humeral prosthesis upper part has a recess in a face thereof that faces toward the glenoid mounted prosthesis in use and which receives a coupling part of the head component.

Preferably each of the first and second head components has collet fingers as a said coupling part and the recess has a shoulder therein with which the collet fingers co-operatively engage in use. In one preferred embodiment a said first head component and said collet fingers are in a coupling part of the head component that is assembled to an articulating part of the head component bearing the convex articulating surface via an intermediate part having further collet fingers.

In accordance with a further aspect of the invention there is provided a glenoid prosthesis which comprises a head part with a rounded convex articulating surface and a body part for mounting into a glenoid, the body part having a substantially frustoconical taper with an integral screw formation extending terminally therefrom whereby when the prosthesis is fixed in place to the glenoid by the screw formation the taper of the body assists in stabilising the prosthesis.

In accordance with a further aspect of the invention there is provided a glenoid prosthesis which comprises a head part with a concave articulating surface and a base part for mounting into a glenoid, the base part having a tray or plate shaped form with an integral screw formation extending from the underside thereof to fix the prosthesis in place to the glenoid, the base part being coupled to the head part, the prosthesis further having an intermediate annular part with lugs to project into the glenoid for stabilisation.

The glenoid prosthesis of these latter aspects may be used as part of a modular system for shoulder joint arthroplasty combined with any of the aforementioned proximal humeral prostheses.

### BRIEF DESCRIPTION OF THE DRAWINGS

Preferred embodiments of the present invention will now be more particularly described, by way of example, with reference to the accompany drawings.

FIGS. 1 to 6 are perspective views of reverse and natural anatomy prostheses for the proximal humerus.

FIGS. 1A and 1B are, respectively, a perspective view and a longitudinal sectional view of a first reverse anatomy type proximal humeral prosthesis for use with a Bayley-Walker™ glenoid screw prosthesis or the like, the latter being shown assembled to the proximal humeral prosthesis in FIG. 2.

FIGS. 3 to 6 show a natural anatomy type proximal humeral prosthesis having a separable ball head component, with FIGS. 3 and 4 being perspective views of the prosthesis during assembly and FIG. 5 being a longitudinal sectional view and FIG. 6 being a perspective view of the prosthesis as assembled.

FIGS. 7A and 7C are perspective views, the former disassembled and the latter assembled, of a prosthesis for resurfacing of the glenoid when damage to the glenoid cavity is substantial but not extreme and the soft tissues of the rotator cuff are in good working order. FIG. 7B is a longitudinal sectional view of the assembled prosthesis and FIG. 8 is a perspective view corresponding to FIG. 7C but of the assembled glenoid prosthesis in use together with the proximal humeral prosthesis of FIG. 8.

FIGS. 9A, 9B and 9C are, respectively: a perspective view of a prosthesis for mounting to the glenoid for use when damage to the glenoid is minimal (suitable for use after the damage to the rotator cuff mechanism deteriorates to the extent where the component of FIG. 7, is no longer able to provide stability in the joint) during assembly to a proximal humeral prosthesis; a longitudinal sectional view thereof as assembled to the proximal humeral prosthesis; and a perspective view thereof as assembled to the proximal humeral prosthesis.

FIGS. 10A, 10B, 11A and 11B are perspective views of a prosthesis for repairing the glenoid when the glenoid cavity is severely damaged. FIGS. 10A and 10B show a first component of the prosthesis, being a shell to mount to the glenoid region. FIGS. 11A and 11B show the first component together with a second, the second component being an insert that nests within the first component and which defines the replacement glenoid articulating surface. In FIG. 11B the second component is shown cemented in place within the first component as it would be once the first component has been secured to the scapula's prostheses by the bolts or screws.

FIGS. 12, 13 and 14 illustrate a prosthesis assembly for the proximal humerus. FIG. 12 is a perspective view of a proximal humeral prosthesis similar to that of FIG. 9A whereof the upper part and the stem that extends into the humerus are integral with each other and where the separate head component having the articulating surface is not attached. FIG. 13 is a disassembled view of the proximal humeral prosthesis and of a ball-shaped head component and a modular connector neck which couples the ball to the upper part of the proximal humeral prosthesis. FIGS. 14A and 14B are longitudinal sectional and perspective views respectively of the shaft component, modular neck and ball element assembled together.

### DESCRIPTION OF PREFERRED EMBODIMENTS

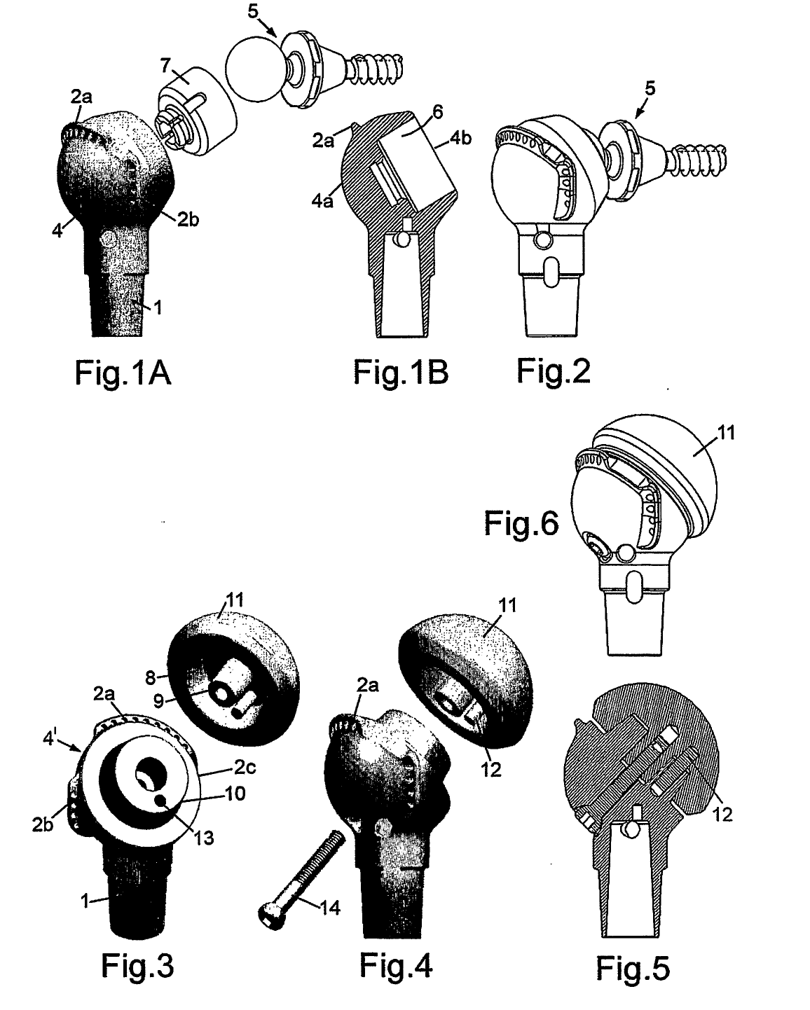

The device illustrated in FIGS. 1 to 6 is a prosthesis for the proximal humerus or rather is an upper part and head component of a modular prosthesis for the proximal humerus and which has a tubular shank 1 to receive an elongate stem component (not shown), if needed, that extends into the core of a resected humerus. Reference is made here to the Applicant's own earlier UK Patent Application No. GB 2370041 the content of which is incorporated herein by reference and that discloses the modular construction of a long bone prosthesis having a head portion with a separable stem component.

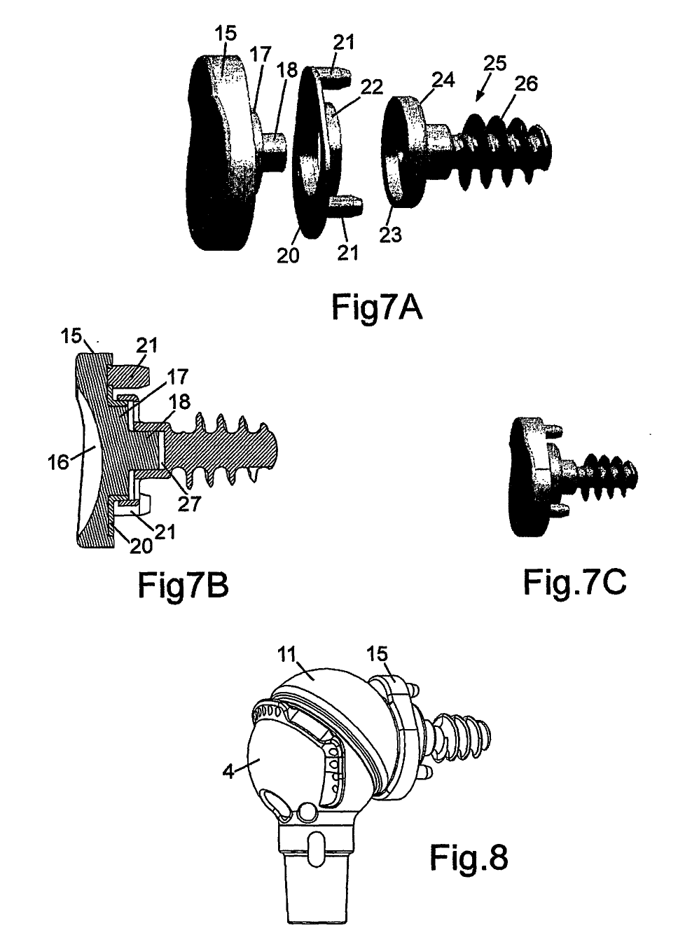

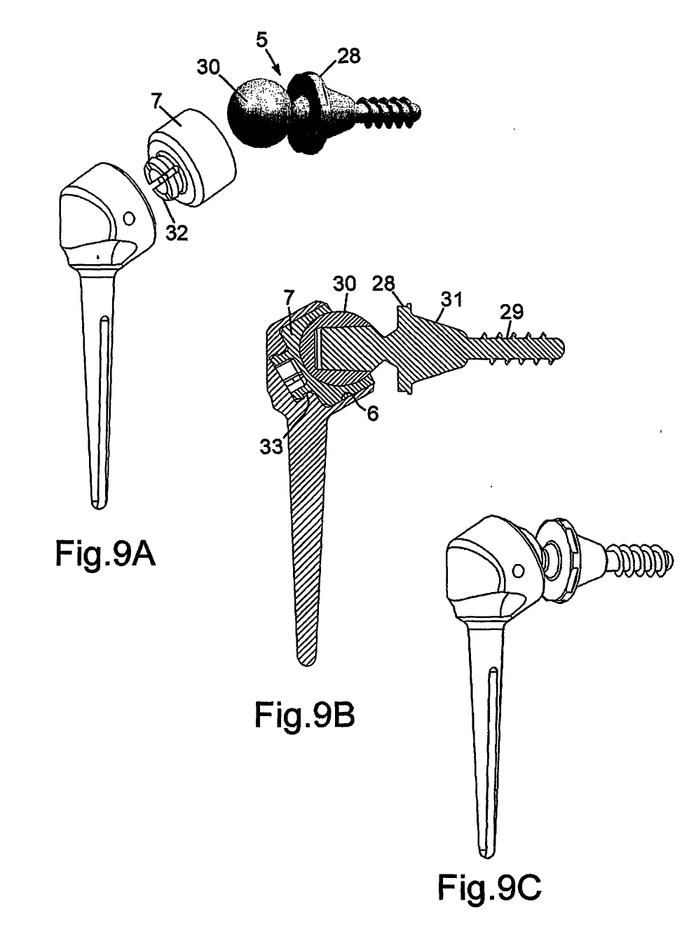

In the proximal humeral prosthesis illustrated in FIGS. 1A, 1B and 2 this is adapted to provide a reverse anatomy type shoulder joint. As shown in FIGS. 1A and 2, it is suited for use in combination with a Bayley-Walker™ glenoid screw prosthesis 5 or similar, such as is discussed later with reference to FIG. 9A, where the ball of the ball-and-socket shoulder joint is provided on the glenoid prosthesis component 5 rather than on the humeral prosthesis component. The proximal humeral prosthesis of FIGS. 1A, 1B and 2 comprises a moulding defining shank 1 and an upper part 4 that corresponds to the upper end of the humerus. The upper part 4 has a convex rounded outer surface 4a and at its inner face 4b that faces toward the glenoid prosthesis 5 has a deep and broad central recess 6 to accommodate a head component 7. Head component 7 has a concave articulating surface for articulating with the ball of the glenoid prosthesis 5. This head component 7 and its manner of mounting to central recess 6 is further detailed with reference to FIG. 9 below.

The rounded outer surface 4a of the proximal humeral prosthesis upper part 4 is configured to correspond to the proximal humerus upper end that it replaces and is uniquely provided with an arrangement of raised flanges/fins 2a, 2b, 2c that are configured to provide secure points of attachment for the muscles/ligaments of the shoulder joint. These flanges 2a, 2b, 2c are perforated at intervals along their length to facilitate initial attachment and then in-growth of the muscles/ligaments/tendons, and their configuration suits the anatomical arrangement of the primary muscles/ligaments/tendons. A first length 2a extends transversely of the axis of the shaft 1 and associated stem to anchor against forces for lifting of the humerus/arm and a second length 2b extends at an angle to the first which is more aligned with the shaft 1 to anchor against forces for rotation of the humerus/arm about its axis. The first length of fin or flange 2a is separated from the second length 2b by a gap for passage of nerves or other tissues. A third length of fin or flange 2c extends down the opposite side of the outer surface 4a substantially parallel to the second length 2b but is not separated from the first length 2a by a gap.

Turning to FIGS. 3 to 6 these show a variant of the proximal humeral prosthesis which unlike that of FIGS. 1A, 1B and 2 is adapted to mimic the natural anatomy of the shoulder joint and has a separable ball type head component 11 defining a generally hemispherical convex articulating surface that is push-fitted to the upper part 4′ of the proximal humeral prosthesis. The underside of the head component 11 has a cavity 8 with a central projection 9, where the cavity 8 fits over a raised annular wall 10 of the proximal humeral prosthesis upper part 4′. A separate smaller projection 12 in the cavity 8 of the head component 11 keys into a socket 13 in the annular wall 10 to prevent rotation of the ball component 11 when mounted in place. A bolt 14 extends up through an aperture in the outer face 4a of the upper part 4′ of the proximal humeral component up through the recess defined by the annular wall 10 and threadedly engages with the projection 9 of the head component 11.

The shoulder joint prosthesis illustrated in FIGS. 7A, 7B, 7C and 8 is a glenoid resurfacing prosthesis that is of modular construction, having three separate parts. A head part 15 defines an artificial glenoid cavity surface 16 and has on its underside a shallow recess having a raised central portion 17 with at its centre a protruding stub 18. The glenoid resurfacing head part 15 is assembled into an intermediate component 19 that is generally annular in shape having an annular plate portion 20 that is shaped to conform to the underside of the head part 15 and which has on its rear face a radially spaced apart set of three lug projections 21, the purpose of which is to provide torsional stability and improve primary fixation. The rear face of the annular plate portion 20 is suitably HA coated.

A raised annular wall 22 protrudes from the rear face of the metal shallow tray/plate portion 20 and slides and fits into a raised annular wall 23 on the forward face of the head 24 of a screw component 25 of the prosthesis. The head 24 of the screw component 25 with its raised annular wall 23 can be regarded as a tray structure. The alternate face of the screw component 25 has an HA coated screw threaded shank 26 extending therefrom and which is screwed into the bone of the glenoid cavity. The central stub 18 of head part 15 seats into a central recess 27 in the screw component 25.

Turning to FIG. 9A, this shows a simpler prosthesis 5 for the glenoid, having the form of a screw with a threaded screw shank 29 to engage into the bone of the glenoid cavity and where the head part of the screw is formed as a ball 30 to co-operatively engage with a concave articulating/bearing surface.

Between the screw threaded shank 29 and the ball 30 head of the FIG. 9 glenoid screw prosthesis is an intermediate portion comprising a plate/shoulder 28 that is circular in plan centred on the axis of the screw thread shank 29 and with a transition from the plate portion 28 to the shank 29 that is uniquely a substantially frusto-conical form 31 tapering toward the shank 29. This frustoconical transition 31 enables the glenoid screw prosthesis to be screw mounted to the glenoid surface to a substantial depth with a reduction in fragmentation of the exposed glenoid face by the screw threaded shank 29 and where the frustoconical surface 31 has a stabilising effect so that when forces are applied to the joint the damage on the remaining glenoid bone is reduced. The frustoconical transition 31 is suitably HA coated.

In FIGS. 9A, 9B and 9C the concave articulating/bearing surface with which the ball 30 of the glenoid mounted prosthesis co-operates is formed in a socket defined in a head component 7 of a proximal humeral prosthesis and where the ball 30 snap fits into the socket. The proximal humeral prosthesis head component 7 illustrated is usable with a proximal humeral prosthesis of the modular separable upper part and stem type shown in FIG. 1 or of the integrated upper part and stem type shown in FIGS. 9A to 9C and has co-operative engaging means in the form of collet fingers 32 to snap-fit engage with a shoulder 33 defined in a central recess 6 of the proximal humeral prosthesis upper part.

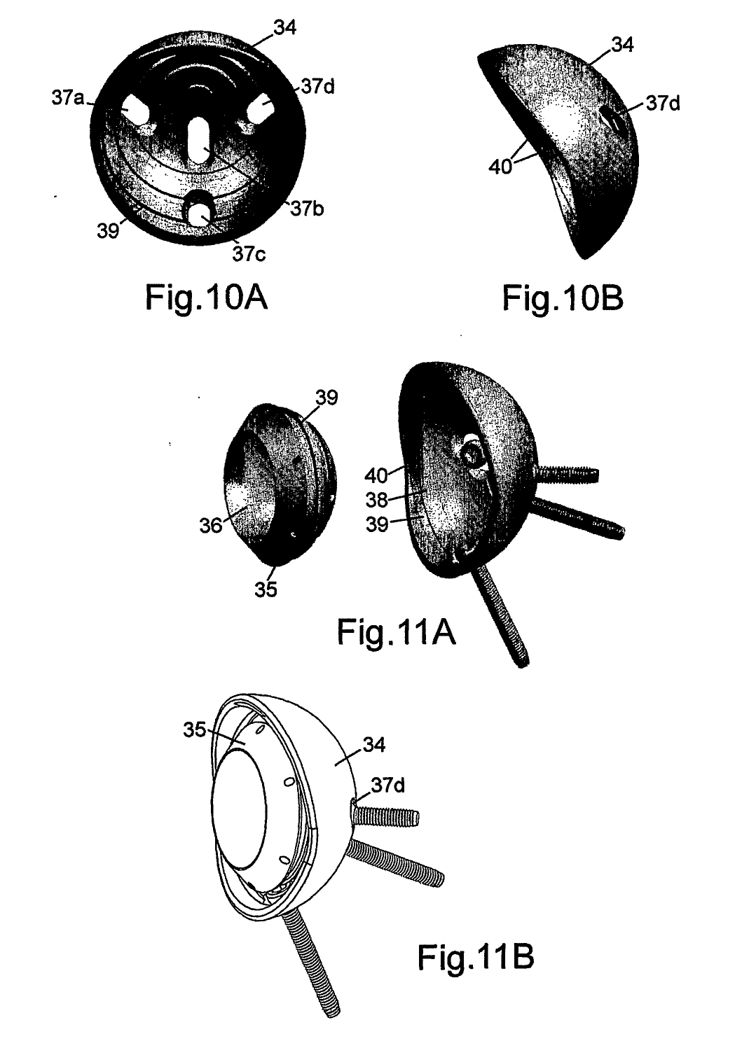

FIGS. 10A, 10B, 11A and 11B show a new glenoid prosthesis assembly that is distinct from presently available prostheses, being specially adapted for use on shoulder joints where the glenoid has become severely damaged/degenerated.

This prosthesis comprises a rigid substantially hemispherical shell-shaped first component 34 that is adapted to be screw mounted/bolted to the glenoid region of the scapula; and a corresponding smaller cup or shell-shaped second component 35 that is adapted to be mounted into the cavity of the first shell shaped component 34. A concave inner surface 36 of the second component defines the articulating surface for articulating with the humeral head.

The first component 34 is shaped to suit the normal anatomy and relative positions of the glenoid, acromion and coracoid processes of the scapula. It preferably is formed of metal. Where screws are used as the cementless fixing means, these suitably are positioned to enter the base of the spine of the scapula in addition to halfway up the spine, to enter the acromion process, the coracoid process, halfway up the coracoid process and to also enter the inferior edge of the glenoid. Slotted screw holes 37a-d are provided on the shell to facilitate adjustment of screw position to best suit the configuration of the relevant anatomy on the patient.

The first component 34 is substantially hemispherical in shape but is less than a hemispherical shape with anterior and posterior reliefs 40 to provide for optimum placement of the second component 35 within the first component 34 for an increased range of motion of the shoulder joint than would be allowed by a purely hemispherical shape with no reliefs.

The first component 34 is suitably coated in titanium plasma spray and surface coated with HA to promote knitting with the patient's bone. The convex rear surface of the second component 35 and the concave internal surface 38 of the first component 34 are each provided with grooves 39 to allow for flow of bonding cement that is used to bond together the first and second components of the prosthesis.

The second component 35 may be of captive type (ie with the ball of the head component snap fitting therewithin) or non-captive depending on whether it is desired to have a constrained or unconstrained joint. Where it is adapted to be for captive joint usage it is suitably also relieved like the first component 34 to allow for a suitable range of motion of the constrained joint.

Suitably a range of different shell sizes are provided to allow for differences in the anatomical relationship of the glenoid, coracoid and acromion processes of different patients' scapulae.

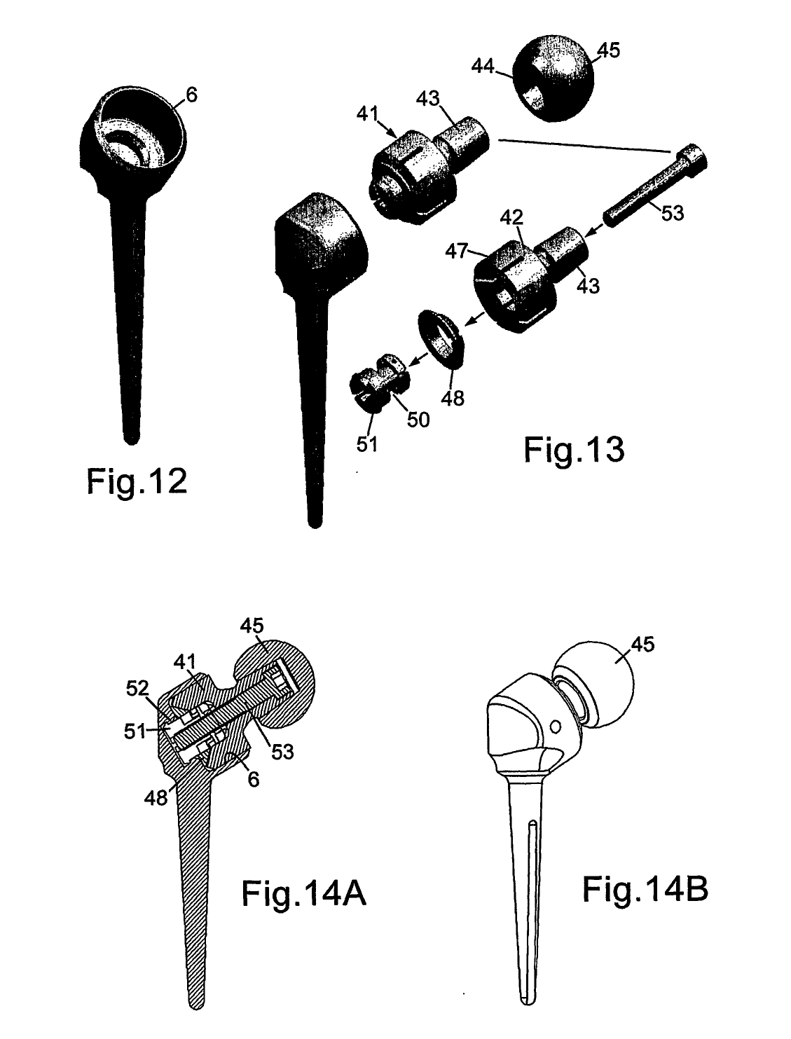

Turning to FIG. 12 this illustrates a substantially conventional proximal humeral prosthesis component of the type having a tapered stem to extend into the humerus and having an integral upper part with a recess 6 to receive a cup-shaped head component 7 to, in turn, receive a ball of a glenoid mounting prosthesis (see also FIG. 9). This proximal humeral prosthesis is, with the cup-shaped head component 7, an existing product that is specifically designed to co-operate with a ball-type glenoid component.

The configuration of the prosthesis assembly comprising the socket on the proximal femoral component and ball on the glenoid component is a reversal of the natural anatomical configuration where the ball is present on the proximal femur and received in the socket defined by the glenoid cavity. For some conditions this is acceptable but for others it is better for the prosthesis configuration to be a natural one.

Referring to FIGS. 13, 14A and 14B, a modular neck component 41 is provided to enable conversion of the head of the proximal femoral prosthesis from defining a socket to instead defining a ball.

The neck component 41 comprises four separate subcomponents as shown in FIG. 13. The first subcomponent comprises a body 42 with a neck portion 43 at one end to mount into a socket 44 of ball 45 and at its other end has a wide diameter collet portion 47 to co-operatively engage with and hold in place a frustoconical washer component 48 and which in turn clamps around and holds a further smaller collet finger subcomponent 50. The further collet finger component 50 has terminal radial flanges 51 on the ends of the collet fingers and is adapted to snap fasten into and hook behind inner socket shoulder 52 in the rear of the major socket/recess 6 of the upper part of the proximal humeral component. A further component of the modular neck assembly is a bolt/compression screw 53 that passes axially through each of the other three components and which screw-threadedly engages with the collet finger component 50 and when fully tightened, screw 53 reaches the level of the radial flanges 51, supporting them and preventing them from disengaging from behind the inner socket shoulder 52.

The neck component 41 provides a modular head which can operate as a constrained joint and the changed head to neck length ratio improves the range of motion available and reduces the risk of dislocation by tightening the ligaments/muscles. The neck also allows for a variable length offset, where needed, to further extend out to compensate for slackness in the ligaments/muscles, providing more controlled muscle function and thereby improving mobility.

The modular ball-shaped head component 41, 44 may also be used with the proximal humeral prosthesis upper part 4 of FIGS. 1A, 1 B and 2, enabling its use as an alternative natural anatomy variant to the natural anatomy variant of FIGS. 3 to 6, giving even greater versatility and choice of arrangement to the surgeon to enable him to tailor the prosthesis assembly to best suit the needs of the individual patient.

1. A glenoid prosthesis assembly for use in shoulder joint arthroplasty comprising a first component to be affixed to a scapula by fixing means, and a smaller second component adapted to nest within the first component and having an articulation surface for articulating with a humeral head, the first component being over-sized relative to the glenoid surface of a scapula whereby the first component may be first affixed to at least two of the glenoid, acromion and coracoid processes of the scapula in use by the fixing means and the second component may be cemented in place within the first component allowing initial adjustability in the poise of the second component relative to the first component.

2. A glenoid prosthesis assembly as claimed in claim 1, wherein at least one of the first and second components has on its surface that mounts to the other component grooves to facilitate cement distribution for cementing the second component in place.

3. A glenoid prosthesis assembly as claimed in claim 1 or 2, wherein the first cup-shaped component is affixed in use to each one of the glenoid, acromion and coracoid processes of the scapula and preferably for this purpose has at least one respective screw/bolt hole therethrough located to correspond to the relative location of each of the glenoid, acromion and coracoid processes of the scapula.

4. A modular proximal humeral prosthesis system for use in shoulder joint arthroplasty which comprises an upper proximal humeral prosthesis part for articulation with a glenoid mounted prosthesis and a proximal humeral prosthesis stem to fit into a resected humerus or even replace the shaft of the humerus, the upper part being demountably mounted to the stem in use, there being a range of alternative upper parts whereof: a first upper proximal humeral prosthesis part has thereon or coupled thereto in use a head component that articulates with a glenoid mounted prosthesis and defining a convex humeral head articulating surface to co-operate with a glenoid mounted component whereof the articulating surface is concave (anatomical configuration); and a second upper proximal humeral prosthesis part which has thereon or coupled thereto in use a head component that articulates with a glenoid mounted prosthesis and defining a concave articulating surface to co-operate with a glenoid mounted component whereof the articulating surface has a convex rounded form, whereby the proximal humeral prosthesis may be changed between anatomical and reverse of anatomical configurations.

5. A modular proximal humeral prosthesis for use in shoulder joint arthroplasty to articulate with a glenoid mounted prosthesis and which comprises an upper part, a head component that assembles to the upper part and articulates with the glenoid mounted prosthesis, and a stem to fit into a resected humerus, the upper part of the prosthesis being formed to replace substantially the whole of the upper end of a proximal humerus including the neck and one or both tubercles, having a substantially hemispherical or rounded outer form with a formation, suitably a recess or projection, at an inner face thereof that faces in use toward the glenoid mounted prosthesis, the formation fitting in use to a coupling part of the head component.

6. A modular proximal humeral prosthesis as claimed in claim 4 or claim 5, having a projecting flange or fin arrangement on the upper part thereof to which soft tissues of the shoulder may attach and anchor.

7. A modular proximal humeral prosthesis as claimed in claim 6, wherein the flange or fin arrangement is configured to provide a first length that extends transversely of the axis of the stem and at least a second length that extends at an angle to the first length which is more aligned with the stem to provide anchoring against forces in more than one axis of articulation of the humerus at the shoulder.

8. A modular proximal humeral prosthesis as claimed in claim 7, wherein the first length of fin or flange is configured to anchor against forces for lifting of the humerus/arm and the second length of fin or flange is configured to anchor against forces for rotation of the humerus/arm about its axis.

9. A modular proximal humeral prosthesis as claimed in claim 8 wherein the first length of fin or flange is separated from the second length of fin or flange by a gap for passage of nerves or other tissues.

10. A modular proximal humeral prosthesis as claimed in any of claims 5, 6, 7, 8 or 9, wherein the head component that articulates with the glenoid mounted prosthesis defines a convex humeral head articulating surface.

11. A modular proximal humeral prosthesis as claimed in any of claims 5, 6, 7, 8 or 9, wherein the head component that articulates with the glenoid mounted prosthesis defines a concave articulating surface to co-operate with a glenoid mounting component whereof the articulating surface has a convex rounded form.

12. A modular proximal humeral prosthesis for use in shoulder joint arthroplasty to articulate with a glenoid mounted prosthesis and which comprises an upper part and a stem to fit into a resected humerus, the upper part of the prosthesis being formed to replace substantially the whole of the upper end of a proximal humerus including the neck and one or both tubercles, having a projecting flange or fin arrangement on the upper part thereof to which soft tissues of the shoulder may attach and anchor.

13. A modular proximal humeral prosthesis system for use in shoulder joint arthroplasty to articulate with a glenoid mounted prosthesis and which comprises an upper part and a stem to fit into a resected humerus, and a head component that assembles to the upper part and articulates with the glenoid mounted prosthesis, there being a range of alternative head components whereof: a first head component has a convex humeral head articulating surface to co-operate with a glenoid mounted component whereof the articulating surface is concave (anatomical configuration); and a second head component has a concave articulating surface to co-operate with a glenoid mounted component whereof the articulating surface has a convex rounded form (reverse of anatomical configuration),
whereby the proximal humeral prosthesis may be changed between anatomical and reverse of anatomical configurations.

14. A modular proximal humeral prosthesis system as claimed in claim 4 or 13, wherein the proximal humeral prosthesis upper part has a recess in a face thereof that faces toward the glenoid mounted prosthesis in use and which receives a coupling part of the head component.

15. A modular proximal humeral prosthesis system as claimed in claim 14 as dependent on claim 13, wherein each of the first and second head components has collet fingers as a said coupling part and the recess has a shoulder therein with which the collet fingers co-operatively engage in use.

16. A modular proximal humeral prosthesis system as claimed in claim 13, or 14 or 15 as dependent on claim 13, wherein the head component is a said first head component and has said collet fingers or other co-operative engagement means in a coupling part of the head component that is assembled to an articulating part of the head component bearing the convex articulating surface via an intermediate neck part having further collet fingers or other co-operative engagement means.

17. A modular proximal humeral prosthesis system as claimed in claim 14, 15 or 16 as dependent on claim 13, wherein the head component is a said first head component having a convex humeral head articulating surface further in combination with the glenoid prosthesis assembly of any of claims 1 to 3 as the glenoid mounted component.

18. A glenoid prosthesis which comprises a head part with a rounded convex articulating surface and a body part for mounting into a glenoid, the body part having a substantially frustoconical taper with an integral screw formation extending terminally therefrom whereby when the prosthesis is fixed in place to the glenoid by the screw formation the taper of the body assists in stabilising the prosthesis.

19. A glenoid prosthesis which comprises a head part with a concave articulating surface and a base part for mounting into a glenoid, the base part having a tray or plate shaped form with an integral screw formation extending from the underside thereof to fix the prosthesis in place to the glenoid, the base part being coupled to the head part, the prosthesis further having an intermediate annular part with lugs to project into the glenoid for stabilisation.

20. A glenoid prosthesis as claimed in claim 18 or 19 as part of a modular system for shoulder joint arthroplasty and further comprising the prostheses of any of claims 1 to 17.
